(19) United States
(12) Patent Application Publication
(10) Pub. No.: US 2024/0341847 A1
(43) Pub. Date: Oct. 17, 2024

(54) AN APPARTUS AND METHOD FOR FRACTIONAL ABLATIVE TREATMENT OF TISSUE

(71) Applicant: IPG PHOTONICS CORPORATION, Marlborough, MA (US)

(72) Inventors: Dilip Paithankar, Waltham, MA (US); Ilya Yaroslavsky, N. Andover, MA (US); Gregory Altshuler, Lincoln, MA (US); Valeriya Arkhipova, Fyazino (RU); Valentin Tyrtyshnyy, Fryazino (RU); Daniil Myasnikov, Fryazino (RU); Igor Larionov, Fryazino (RU); Alexander Gulyashko, Fryazino (RU); Mikhail Nosov, Fryazino (RU)

(73) Assignee: IPG PHOTONICS CORPORATION, Marlborough, MA (US)

(21) Appl. No.: 18/683,860

(22) PCT Filed: Sep. 2, 2022
(86) PCT No.: PCT/US22/42478

§ 371 Date: Feb. 15, 2024

Related U.S. Application Data
(60) Provisional application No. 63/240,119, filed on Sep. 2, 2021.
(60) Provisional application No. 63/243,489, filed on Sep. 13, 2021.

(51) Int. Cl. A61B 18/22; A61B 18/00; A61B 18/20

(52) U.S. Cl. CPC A61B 18/22; A61B 2018/0047; A61B 2018/00559; A61B 2018/00577; A61B 2018/00589; A61B 2018/00702; A61B 2018/00726; A61B 2018/2035; A61B 2018/20351; A61B 2018/2065

(57) ABSTRACT

A device for performing treatment of biological tissue that includes a laser system configured to provide a laser beam having a wavelength within a range of 3.0 microns (μm) to 3.25 μm inclusive and a spot size within a range of 10 μm to 45 μm inclusive, and a controller coupled to the laser system and configured to scan the laser beam over the biological tissue in an injury pattern, the injury pattern having a pitch that is sized to be in a range of 0.1 mm to 1 mm inclusive.

### RELATED APPLICATIONS

The present application claims priority to U.S. Provisional Application Ser. No. 63/240,119, filed on Sep. 2, 2021 and U.S. Provisional Application Ser. No. 63/243,489, filed on Sep. 13, 2021, each titled “APPARATUS AND METHOD FOR FRACTIONAL ABLATIVE TREATMENT OF TISSUE,” the contents of which are herein incorporated by reference in their entirety.

### BACKGROUND

### Technical Field

The technical field relates generally to fractional ablative treatments of biological tissue using laser energy.

### Background Discussion

Fully ablative biological tissue treatment methods using directed laser energy work very well for treatments such as skin resurfacing which yield improvement in wrinkles or lax skin, but can have significant long-lasting side effects that make them unattractive. In response, fractional laser ablative treatments have been developed. During such a treatment, columns of injury separated by areas of undamaged skin are created. Such columns can be created with a scanner or a microlens or diffraction optics. This leads to an improvement in skin attributes such as texture, fine lines, wrinkles, scars, and abnormal pigmentation. The big advantage is that the downtime and side effects are reduced in severity and relatively short-lived compared to a fully ablative treatment due to rapid healing and the less than full ablation.

However, conventional fractional ablative treatments have lower efficacy than fully ablative treatments. The primary reason for this is that a substantial portion of the skin is untreated. In other words, the skin area with “controlled injury” is low. Furthermore, the social downtime for conventional fractional ablative treatments is about 3-15 days and ideally needs to be shortened, e.g., 1-3 days.

Aspects and embodiments are directed to methods and devices for treating biological tissue.

In accordance with an exemplary embodiment, there is provided a device for performing treatment of biological tissue that includes a laser system configured to provide a laser beam having a wavelength within a range of 3.0 microns (μm) to 3.25 μm inclusive and a spot size within a range of 10 μm to 45 μm inclusive, and a controller coupled to the laser system and configured to scan the laser beam over the biological tissue in an injury pattern, the injury pattern having a pitch that is sized to be in a range of 0.1 mm to 1 mm inclusive.

In one example, the spot size is within a range of 30 μm to 45 μm inclusive.

In one example, the laser system is configured to generate pulsed radiation such that a radiant exposure (RE) per pulse is within a range of 30 J/cm² to 6000 J/cm² inclusive. In a further example, the RE per pulse is within a range of 100 J/cm² to 4000 J/cm² inclusive.

In one example, the injury pattern is an array of spots or lines.

In one example, the injury pattern is an array of spots on a surface of the biological tissue having a number density within a range of 100 spots/cm² to 10000 spots/cm² inclusive.

In one example, the laser system is configured to generate pulsed radiation and the injury pattern includes ablation columns, the ablation columns having a column density defined as a number of columns per square centimeter of biological tissue, and the column density having a maximum value of 10000, 7500, 6500, 5000, 4000, 3500, 3000, 2500, 1800, 1700, 1600, 1500, 1400, 1300, 1000, and 500 for ablation depths of 25, 50, 100, 200, 250, 300, 350, 450, 550, 650, 750, 900, 1000, 1500, 2000, and 3000 μm respectively.

In one example, the laser system is configured to generate pulsed radiation and the injury pattern includes ablation columns, the ablation columns having a column density defined as a number of columns per square centimeter of biological tissue, and the column density having a minimum value of 1300, 1200, 1100, 1000, 1000, 1000, 1000, 1000, 1000, 1000, 900, 800, 700, 600, 500, and 300 for ablation depths of 25, 50, 100, 200, 250, 300, 350, 450, 550, 650, 750, 900, 1000, 1500, 2000, and 3000 μm respectively.

In a further example, the column density has an intermediate value for the column density and ablation depth that is obtained by interpolating between adjacent column density and ablation depth values.

In one example, the laser system is configured to generate pulsed radiation, the injury pattern is an array of spots, and the controller is further configured to scan the laser beam such that a radiant exposure (RE) per pulse is decreased on spots positioned near one or more edges of the array.

In one example, the injury pattern is an array of spots and the controller is further configured to scan the laser beam such that a number density of spots is lower near one or more edges of the array.

In one example, each spot in the array of spots and each line in the array of lines has an ablation depth within a range of 25 μm to 3000 μm inclusive.

In one example, the laser system is configured to generate pulsed radiation such that each pulse has a peak power within a range of 0.1 W to 50 W inclusive.

In one example, the laser beam is incident on a surface of the biological tissue with a spot having an intensity profile that is a quasi-Gaussian profile, a flat-top profile, or a Bessel-Gauss profile.

In one example, controller is further configured to control or modulate at least one laser parameter of the laser system. In one example, a laser source of the laser system is configured to operate in a pulsed mode and the at least one laser parameter includes a pulse duration within a range of 1 microsecond (μs) to 250 milliseconds (ms) inclusive, and a duty cycle within a range of 5% to 90% inclusive.

In one example, the laser beam has an M² value in a range of 1.0 to 1.5 inclusive. In a further example, the laser beam has an M² value in a range of 1.0 to 1.3 inclusive.

In one example, the laser system comprises a laser module comprising at least one laser source, a difference frequency generator located within a handpiece, an optical focusing system located within the handpiece and configured to focus the laser beam to the spot size, and an optical fiber coupled to the laser module and the difference frequency generator.

In one example, the difference frequency generator is an optical parametric oscillator (OPO).

In one example, a laser beam of laser radiation generated from the OPO is directed onto a treatment area of the biological tissue, the laser beam configured to perform tissue ablation and coagulation.

In one example, at least a portion of laser radiation emitted from the OPO is directed back to the laser module.

In one example, the device further includes a scanner located within the handpiece.

In one example, the laser module comprises two diode pumped fiber laser sources. In a further example, a single mode (SM) fiber delivers laser radiation emitted from each of the two diode pumped fiber laser sources into a multiplexer where the laser radiation is combined and delivered to the difference frequency generator by the optical fiber. In one example, laser radiation emitted from each of the two diode pumped fiber laser sources is mixed and delivered to the difference frequency generator by the optical fiber.

In accordance with another exemplary embodiment, there is provided a method of conducting an ablative laser treatment on biological tissue that includes generating a laser beam having a wavelength within a range of 3.0 microns (μm) to 3.25 μm inclusive and a spot size in a range of 10 μm to 45 μm inclusive, and creating an injury pattern on the biological tissue with the laser beam.

In one example, the spot size is within a range of 30 to 45 microns inclusive.

In one example, the injury pattern includes ablation columns and the laser beam delivers pulsed laser radiation, the ablation columns having a column density defined as a number of columns per square centimeter of biological tissue, and the column density having a maximum value of 10000, 7500, 6500, 5000, 4000, 3500, 3000, 2500, 1800, 1700, 1600, 1500, 1400, 1300, 1000, and 500 for ablation depths of 25, 50, 100, 200, 250, 300, 350, 450, 550, 650, 750, 900, 1000, 1500, 2000, and 3000 μm respectively.

In one example, the injury pattern includes ablation columns and the laser beam delivers pulsed laser radiation, the ablation columns have a column density defined as a number of columns per square centimeter of biological tissue, and the column density having a minimum value of 1300, 1200, 1100, 1000, 1000, 1000, 1000, 1000, 1000, 1000, 900, 800, 700, 600, 500, and 300 for ablation depths of 25, 50, 100, 200, 250, 300, 350, 450, 550, 650, 750, 900, 1000, 1500, 2000, and 3000 μm respectively.

In another example, the method includes intermediate values for the column density and ablation depth by interpolating between adjacent column density and ablation depth values.

In accordance with another exemplary embodiment, a laser system configured to provide laser radiation for performing treatment of biological tissue is provided that includes a laser module comprising at least one laser source, an optical focusing system configured to focus a laser beam of laser radiation generated by the at least one laser source into a spot size, a handpiece configured to direct the laser beam onto the biological tissue in an injury pattern, a difference frequency generator located within the handpiece, and an optical fiber coupled to the laser module and the difference frequency generator.

In one example, the difference frequency generator is an optical parametric oscillator (OPO). In one example, at least a portion of laser radiation emitted from the OPO is directed back to the laser module. In one example, the laser module comprises two diode pumped fiber laser sources. In one example, a first of the two fiber lasers is configured to generate laser radiation having a wavelength within a range of 1.00-1.05 μm and a second of the two fiber lasers is configured to generate laser radiation having a wavelength within a range of 1.5-1.6 μm. In one example, the beam spot has a spot size within a range of 10 μm to 45 μm inclusive. In one example, a laser beam of the laser radiation generated by the two fiber lasers has an M² value in a range of 1.0 to 1.5 inclusive. In one example, the laser system further includes a scanner located within the handpiece, the scanner configured to create the injury pattern on the biological tissue. In one example, the optical focusing system is located within the handpiece.

In accordance with another exemplary embodiment, a laser system configured to provide laser radiation for performing treatment of biological tissue is provided that includes a beam of laser radiation having a wavelength within a range of 3.0 microns (μm) to 3.25 μm inclusive and an M² value in a range of 1.0 to 1.5 inclusive, a handpiece configured to direct the beam of laser radiation onto the biological tissue in an injury pattern, and an optical fiber configured to transmit laser radiation generated by a laser source to the handpiece. In one example, the beam of laser radiation has a spot size within a range of 10 μm to 45 μm inclusive. In one example, the optical fiber has a core diameter that is within a range of 10 μm to 90 μm inclusive.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Embodiments disclosed herein may be combined with other embodiments, and references to “an embodiment,” “an example,” “some embodiments,” “some examples,” “an alternate embodiment,” “various embodiments,” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is a graph showing the absorption of water in a wavelength range of 2.5-3.5 microns and one example of a range of operating wavelengths for a laser system in accordance with aspects of the invention;

FIG. 2 is a schematic representation of one example of a microfractional injury pattern in accordance with aspects of the invention;

FIGS. 3A-3C are schematic representations of other examples of microfractional injury patterns in accordance with aspects of the invention;

FIG. 4 is a schematic representation of an array of spots with varying pulse energy in accordance with aspects of the invention;

FIG. 5 is a schematic representation of an array of spots with varying pitch in accordance with aspects of the invention;

FIG. 6 is a graph showing spot density versus scan dimension in accordance with aspects of the invention;

FIG. 7 is a graph showing pulse energy versus scan dimension in accordance with aspects of the invention:

FIG. 8 is a graph showing a maximum number density versus ablation depth in accordance with one or more aspects of the invention;

FIG. 9 is a schematic representation of one example of a laser system in accordance with one or more aspects of the invention;

FIG. 10 shows perspective exterior views of two examples of handpieces in accordance with one or more aspects of the invention;

FIG. 11 is a schematic representation of one example of a handpiece in accordance with aspects of the invention;

FIGS. 12A and 12B are bar charts showing measured TEWL values for treatment and control areas from experiments performed in accordance with aspects of the invention; and

FIG. 13 is a graph showing ablation depth as a function of radiant exposure in accordance with aspects of the invention.

### DETAILED DESCRIPTION

### Overview

As mentioned above, conventional fractional laser ablative treatments have lower efficacy than fully ablative treatments and there remains a need for the ability to improve efficacy by increasing the area treated while not increasing social downtime. In accordance with at least one embodiment, this is accomplished by reducing the spot size. It is to be appreciated that the spot size refers to the beam spot (or beam size) on the surface of the biological tissue. The spot size is about equal to the diameter of the ablative column, which is a major factor in defining the healing time and social downtime. Conventional fractional laser treatment devices have spot sizes that are 120 microns (μm) or higher. In accordance with at least one embodiment, spot sizes on the order of less than 45 μm, and in some instances, on the order of 10-30 μm, are successfully used on biological tissue. These sizes are referred to as “microfractional” as used herein. The small spot size leads to faster healing and allows for a high number density (or coverage rate) with much reduced magnitude and duration of side effects, which leads to higher efficacy than conventional fractional treatments.

In accordance with at least one embodiment, the healing rate from injury is proportional to (l/(spot size)), for the same “coverage rate” (where “coverage rate” is defined as the area damaged/total area), when comparing different spot sizes. The healing rate is in turn inversely proportional to the post-treatment downtime. Thus, with a lower spot size, lower pitch, and a higher number density, a closer to fully ablative-like efficacy can be obtained while reducing downtime, risk, magnitude, and duration of the adverse side effects. In accordance with one embodiment, a laser system is disclosed that is configured to provide a laser beam having a spot size within a range of 10 μm to 45 μm inclusive. As discussed in further detail below, the laser beam in some embodiments also has a wavelength within a range of 3.0 μm to 3.25 μm inclusive. These laser energy wavelengths are able to achieve tissue ablation starting from the skin surface with radiant exposures above the ablation threshold by heating the tissue water to boiling temperature with an optimal coagulation zone width surrounding the ablated zone to regulate the tissue healing and regeneration process. In contrast, wavelengths that are less absorbed are employed in non-ablative laser treatments which heat up the skin tissue, and have peak temperatures that do not reach the boiling point of water, which principally leads to coagulation instead of ablation. Non-ablative fractional treatments have been found to be not very effective for the skin tightening and wrinkle reduction treatments described herein.

According to some embodiments, the fractional ablative treatment on skin can be an array of columns (also referred to as ablation columns) that are substantially perpendicular to skin (i.e., biological tissue). In some embodiments, the injury can also be an array of lines. The lines may also be referred to as grooves. In some embodiments, the lines can be broken up (called “dashes”). In some embodiments, the lines can be put in a unidirectional pattern to maximize efficacy and minimize downtime, for wrinkles or such conditions in a certain direction that result from either muscle movement or Langer's lines in the collagen connective tissue in skin.

According to some embodiments, the ablation depth is a strong function of the radiant exposure (RE), which has been verified by experiments performed by Applicant on ex vivo minipig skin, which is a good model for human skin.

According to some embodiments, the maximum density (number of columns per square cm) is determined in human skin based on acceptable a) pain, and b) side effects from tests performed on humans. In accordance with various embodiments, these results provide the upper limit value or boundary of the density (equivalent to coverage rate) for various ablation depths that correspond to specific radiant exposures for a given wavelength or range of wavelengths.

The methods and systems disclosed herein can be applied to dermatology (skin) and gynecology (vaginal epithelium).

### Wavelength

Conventional lasers used for ablative fractional or non-fractional resurfacing in dermatology and gynecology are CO₂ (having a 10.6 μm wavelength) and Er:YAG (having a 2.94 μm wavelength). Another example also include Er:YSGG (having a 2.79 μm wavelength) in dermatology (one example of a commercial product at this wavelength is Pearl Fractional™, Cutera Inc., Brisbane, CA). In tissue such as skin or vaginal tissue, water is the primary chromophore for the above lasers. The water content in skin or vaginal tissue is typically 70%. The absorption coefficient of water for the above laser wavelengths are: CO₂ (10.6 μm): mua_water 800 cm⁻¹; mua_skin=70% of mua_water:=560 cm⁻¹, Er:YAG, 2.94 μm, mua_water=12,800 cm⁻¹; mua_skin=70% of mua_water=8,960 cm⁻¹, and Er:YSGG, 2.79 μm, mua_water=5,000 cm⁻¹, mua_skin=70% of mua_water=3,500 cm⁻¹, where mua_water absorption coefficient of water, and mua_skin=absorption coefficient of skin. Light from a wavelength of 2.94 μm is very highly absorbed by water (having a water absorption coefficient of about 12,800 cm⁻¹). The radiation at this wavelength is absorbed within a very short depth due to the high absorption coefficient, and the resulting ablation efficiency is very high with a thin coagulation zone outside the ablation zone. With a 10.6 μm wavelength CO₂ laser (having a water absorption coefficient of about 800 cm⁻¹), a thicker coagulation zone is obtained and the treatment is more painful. The thicker zone of coagulation is absorbed by the body over a longer time period, thus increasing the healing time. However, during the longer healing time, there is better regeneration of skin closer to new skin, leading to better cosmetic results, albeit with an elevated chance of side effects such as scarring. In contrast, with the 2.94 μm Er:YAG laser, the treatment is less painful and skin heals more quickly, leading to a reduced risk of scarring but with reduced cosmetic benefit.

One or more embodiments disclosed herein use a wavelength range that combines the advantages of each—lower pain, lower risk of scarring, quicker healing (noted with an Er:YAG laser wavelength), AND increased efficacy (with the CO₂ laser wavelength), and this wavelength range has intermediate absorption coefficients between those obtained with the Er:YAG and CO₂ lasers. In accordance with one embodiment, the water absorption coefficient is within a range of 2100 to 11640 cm⁻¹. These are obtained with the following wavelength ranges: 2.75 μm to 2.85 and 3.0 μm to 3.25 μm. In some embodiments, the wavelength is within a range of 3.0 μm to 3.25 μm inclusive.

According to another embodiment, a wavelength corresponding to a water absorption coefficient that is within a range of 700 cm⁻¹ to 11640 cm⁻¹ inclusive is used. In some embodiments, this includes the CO₂ laser wavelength.

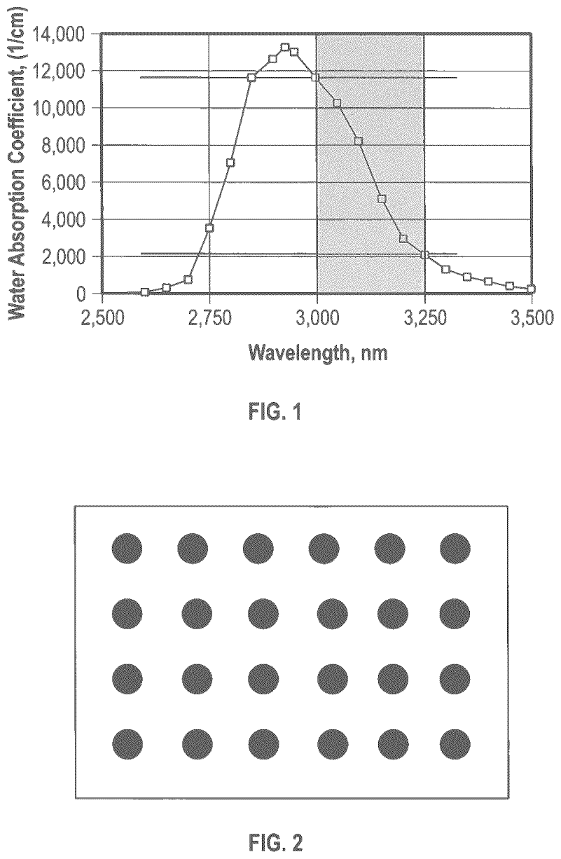

FIG. 1 is a graph showing the absorption of water (liquid) in a wavelength range of 2.5-3.5 μm with an absorption peak at about 2.94 μm. The shaded area in FIG. 1 shows one non-limiting example of a range of operating wavelengths for a laser system in accordance with one embodiment, which in this example is within a range of 3.0 μm to 3.25 μm inclusive. This range is more limited than ranges described in prior literature and is selected in part to avoid the absorption peak at 2.94 μm, which, as discussed above, creates limited coagulation effects. For example, using a laser configured at 2.8 or 2.9 μm wavelengths would create enhanced ablation, but less than desired coagulation. The coagulation zone (e.g., coagulation width) would be less than optimal for the desired cosmetic effect.

According to some embodiments, a laser system configured with a single stage OPO having a 3.05 μm wavelength output and a peak power output within a range of 0.1 W to 50 W inclusive is used. In another embodiment, the peak power output is within a range of 0.1 W to 20 W inclusive. In at least one embodiment, a single stage OPO is configured to output an average power of about 10 W at a 3.05 μm wavelength. This has a water absorption coefficient of about 10,000 cm⁻¹ and has been found to be very efficient in causing ablation. However, in some instances, the coagulation width is considered to be too small (˜20 μm).

In accordance with another embodiment, laser light at a wavelength with a lower absorption coefficient is used for purposes of increasing the penetration depth and causing additional coagulation, which is desirable to achieve skin shrinkage and better cosmetic results. According to some embodiments, a laser source configured with a wavelength of 3.20 μm is generated, which has an absorption coefficient of water around 3,635 cm⁻¹. In one embodiment, such a wavelength is achieved by performing non-linear mixing of 1.56 μm and 3.05 μm to yield 3.20 μm in a second OPO stage. In some embodiments, pulsed radiation is generated, where each pulse has a peak power within a range of 0.1 W to 50 W inclusive. For example, in one embodiment a laser beam exiting the second OPO stage comprises a first wavelength of about 3.05 μm and a first peak power, and a second wavelength of about 3.2 μm and a second peak, power, and a sum of the first and second peak powers is within a range of 0.1 W to 20 W inclusive. In one embodiment, this yielded 10 W of average power. In accordance with certain aspects, the breakdown of power in the two wavelength bands at 20 W total power is ⅔ at 3.05 μm and ⅓ at 3.20 μm.

According to another embodiment, a first laser source is provided that is configured to provide a laser beam having a wavelength within a range of 1.4 μm to 1.6 μm inclusive and a spot size within a range of 10 μm to 45 μm inclusive, and a second laser source is provided that is configured to provide a laser beam having a wavelength within a range of 3.0 μm to 3.25 pin inclusive and a spot size within a range of 10 μm to 45 μm inclusive. According to some embodiments, a treatment method comprises co-locating the spot sizes of the two systems on the surface of biological tissue, operating the two laser systems synchronously or sequentially, and scanning the laser beams of the two systems over the biological tissue in an injury pattern that has a pitch.

### Applications in Dermatology and Gynecology

According to some embodiments, non-limiting examples of dermatology applications where the disclosed systems and methods may be used include:
1. Improvement in superficial lines and wrinkles of biological tissue such as skin.
2. Improvement in deep lines and wrinkles of skin and tightening of lax skin.
3. Improvement in appearance of scars, e.g., acne scars, traumatic injury scars, burn scars.
4. Delivery of drugs into the dermis.
5. Reduction in abnormal undesired pigmentation such as that caused by sun damage.

According to some embodiments, non-limiting examples of gynecology applications where the disclosed system and methods may be used include:
1. Improvement in vaginal laxity, dryness, thin vaginal wall, urinary stress incontinence, dyspareunia, dysuria, sexual function, and other genitourinary symptoms of menopause (GSM).
2. Delivery of drugs such as topically applied hormones through the vaginal epithelium.

### Patterns of Fractional Injury

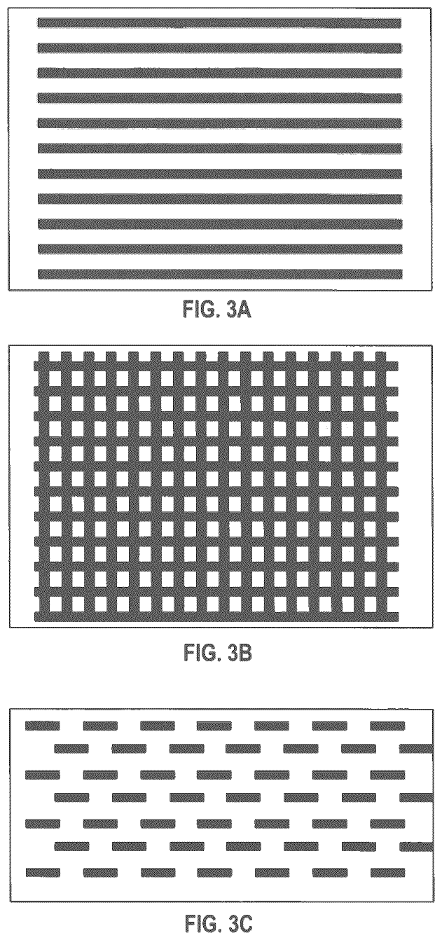

In accordance with certain embodiments, a pattern of fractional injury includes at least one of an array of spots on tissue and lines on tissue. The spots on the tissue may also be referred to as ablative columns. For an array of spots, the array may be square or hexagonal or any other periodic or random pattern. As previously mentioned, the lines may also be referred to as grooves. For lines, the lines may include parallel lines, i.e., lines separated by undamaged skin. In some embodiments the lines are “broken” and are also referred to herein as “dashes.” Both types of patterns are described further below in reference to FIGS. 2 and 3A-3C.

### “Microfractional” Injury Pattern

In ablative fractional treatments, healing occurs from the outer surfaces of the injured columns or grooves. Conventional ablative fractional treatments employ a spot size larger than 120 μm, whereas the methods and systems described herein employ much smaller spot sizes.

A comparison can be performed between the results from low and high diameter columns. The healing rate is principally proportional to the cylindrical surface area of the columns. It can be shown from geometric considerations that the healing rate for a given area is proportional to (1/column diameter). The smaller the diameter, the faster the healing for the same damaged surface area per total surface area. The goal is to minimize the downtime (such as 2-3 days) while approaching the efficacy obtained with fully ablative fractional treatments. The small column diameter can be achieved by using a small spot size (≤45 μm) and as mentioned is defined as “microfractional” for the purposes of this disclosure. The small laser beam spot (spot size) is obtained by focusing the laser beam on the surface of the biological tissue. The diameter of this spot is called the spot size and for a Gaussian beam is defined as the diameter of the circle where the irradiance is (1/e²) or 13.5% of its maximum value. To obtain a given coverage rate, and according to at least one embodiment, it is proposed to use smaller spot sizes combined with a high number density per square cm, which leads to low downtime and higher efficacy. A coagulation zone can also be added at the bottom of the columns and is described in further detail below.

FIG. 2 is a non-limiting example of a microfunctional injury pattern. According to one embodiment, the spot size is in a range of 10 μm to 45 μm inclusive.

In accordance with at least one aspect, it is hypothesized that with microfractional treatment using such small spot sizes, the new skin is true regeneration which means that the skin shows normal collagen and elastin architecture. This is in contrast to abnormal collagen and elastin architecture skin with scars that is seen when larger spots are utilized.

### Desired Wound Closure Time of 1 Day or Less

It is highly desired that the ablative wounds on the skin close as rapidly as possible. This not only leads to better cosmesis but also reduces the probability of infection. The microfractional systems and methods disclosed herein lead to such quick healing with its inherent advantages.

It is hypothesized that transepidermal water loss (TEWL) can be used as a measure of the wound closure. Immediately after microfractional ablative treatment, it is expected that TEWL will increase due to the loss of the skin barrier function at the location of the holes in skin. Once the wound is substantially closed and re-epithelized, the TEWL value is expected to return to close to its baseline value.

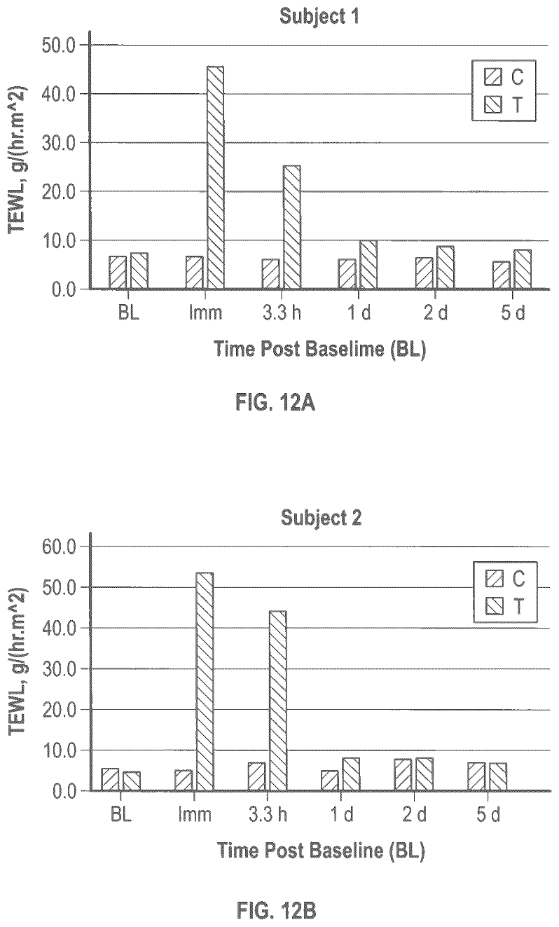

An experiment was conducted to test this hypothesis. A Tewameter® (Courage and Khazaka TM300) and a laser device (equipped with 3.0 μm and 3.25 μm wavelengths, a 50 μm spot size, and a scanner) were used to perform treatments on two subjects using the following parameters:
Treatment was performed on forearm of each subject
Scanned area: 10 mm×10 mm, 50 spots in the area
Pitch, x-direction: 0.5 mm
Pitch, y-direction: 1.0 mm
Pulse energy: 8.6 mJ
Measurements of TEWL: Baseline, Post-Treatment (Immediate, 3-4 h, 1 day, 2 day, 5 days)
   The results are summarized for subjects 1 and 2 in FIGS. 12A and 12B respectively (where C=control, and T=treated area, with the treated results to the immediate right of the control results) with measurement results shown at baseline (BL, before treatment), Immediately post treatment (1 mm, within 10 minutes), 3.3 hours post treatment, (3.3 h), 1 day post treatment (1 d), 2 days post treatment (2 d), and 5 days post treatment (5 d). After treatment, there was an immediate increase (˜1,000%) in TEWL for both subjects, and after 1 day, TEWL of the treated area was close to control (within “noise band”). Thus, with microfractional treatment, the wound closure time is estimated to last less than 1 day. In accordance with one embodiment, similar or shorter closure time can be expected with a smaller (less than 50 μm) spot size.

### Aspect Ratio of Columnar or Line Injury

The aspect ratio for a column injury according to at least one embodiment is defined as the ratio of depth of injury to the diameter of the injury. A range of 0.5 to 100 inclusive is disclosed according to one embodiment. In another embodiment, the range is within 1.0 to 100 inclusive. For lines, the line width is used instead of diameter.

### Lines

In accordance with various embodiments, lines along, perpendicular, or at an arbitrary angle to the natural tension lines of wrinkles or scars are disclosed. One non-limiting example of a fractional pattern with lines is shown in FIG. 3A. In some embodiments, the lines may not be contiguous but broken up within a scan field (“grooves”), as seen in the example shown in FIG. 3C. The depth and pitch are also adjustable. The depth of ablation can be low (as “superficial ablative”) or high (as “deep ablative”. The ablation zone is typically surrounded by a coagulation zone. Instead of ablation, complete coagulation is also an option. According to one embodiment, lines in two directions are also disclosed (e.g., perpendicular to each other), as seen in the example shown in FIG. 313.

### Modulated Injury in a Line

It is contemplated that the spot size will be scanned at a certain velocity across the scan field for the spot to traverse in a line. According to some embodiments, during this scan-time, at least one parameter of the laser system, such as the laser power, is modulated (e.g., via pump laser modulation or other such method). Then, after the one line scan is complete, in some embodiments, the spot will be moved in a direction perpendicular to the direction of motion by a certain distance (i.e., the pitch) and will again be translated as above across the scan field.

In accordance with some embodiments, non-limiting examples of modulating the laser power (i.e., pulsed mode) in a periodic manner include (a) sinusoidal modulation where one or more of minimum power (P_min), maximum power (P_max), and frequency is adjusted, and (b) “square” pattern modulation with different on-time and off-time, and the rise-time and fall-time are about 0.2 ins.

### Laser Parameters to Obtain the Controlled Ablative Injury

In accordance with at least one embodiment, a laser system (described in further detail below) is configured to generate pulsed radiation such that a RE per pulse to the biological tissue is within a range of 30 J/cm² to 6000 J/cm² inclusive. In another embodiment, the radiant exposure (RE) per pulse is within a range of 100 J/cm² to 4000 J/cm². As used herein, the term “radiant exposure” per pulse signifies the energy density or total pulse energy divided by the surface area of a circular (having a diameter that is the same as the spot size) of the spot. In medical and dermatology literature, “fluence” is also a term used to describe this quantity. As used herein, these terms may be used interchangeably. Treatment methods can generally be categorized as either superficial or deep ablation and are outlined below. In accordance with at least one embodiment, each spot or each line in an array has an ablation depth with a range of 25 μm to 3000 μm inclusive.

### Superficial Ablation, Ablation Depth 25-500 μm

Methods of treatment to achieve superficial ablation (i.e., ablation depth range: 25 μm-500 μm) and deep ablation (i.e., 500 μm-3,000 μm, discussed below) in columns are disclosed herein. The wavelengths of the laser used in accordance with one embodiment are 3.05 μm and 3.2 μm. The ablation depth is principally dependent on radiant exposure (RE, J/cm²). As an example, for a beam waist of 34 μm, a 2.0 mJ/pulse (220 J/cm² RE) yields an ablation depth of 200-250 μm. Similarly, a 5 mJ/pulse (551 J/cm²) yields an ablation depth of ˜500 μm. To obtain a certain pulse energy, various power, pulse duration combinations can be used. According to at least one embodiment, the laser power is in a range of 1 W to 20 W. In some embodiments, the pulse duration is in a range of 0.1 to 5 ms. In one embodiment, the laser power is in a range of 2.5 to 5 W for superficial ablation columns. In certain embodiments, such smaller power values allow reproducible pulse energies and smaller (desirable) thermal damage diameter.

### Example—Laser Parameters for Superficial Ablation for a Spot Size of about 34 μm

RE: 100-600 J/cm²
Peak Power range: 1 to 20 W. Preferred: 1.0-5.0 W. More Preferred: 2.0 to 4.0 W.
Pulse energy: 1.0 to 5.0 mJ
Pulse duration: 0.5 ms to 5 ms
   Deep Ablation, Ablation Depth 500 μm-3,000 μm

Methods of treatment to achieve deep ablation (i.e., 500 μm-3,000 μm) in columns are disclosed herein. In some embodiments, the wavelengths of the laser are 3.05 and 3.20 μm. As an example, for a beam waist of 34 μm, a 10 mJ/pulse (1101 J/cm² RE) yields an ablation depth of 700-900 μm. Similarly, a 20 mJ/pulse (2200 J/cm²) yields an ablation depth of ˜1000 μm. To obtain a certain pulse energy, various power and pulse duration combinations can be used. According to at least one embodiment, the laser power is in a range of 10 W to 20 W. In some embodiments, the pulse duration is in a range of 0.1 to 5 ms.

### Example—Laser Parameters for Deep Ablation for a Spot Size of about 34 μm

RE: 500-8800 J/cm2
Peak Power range: 1 to 20 W. Preferred: 10-20 W
Pulse energy: 5.0 mJ-80 mJ
Pulse duration: 0.5 ms to 5 ms

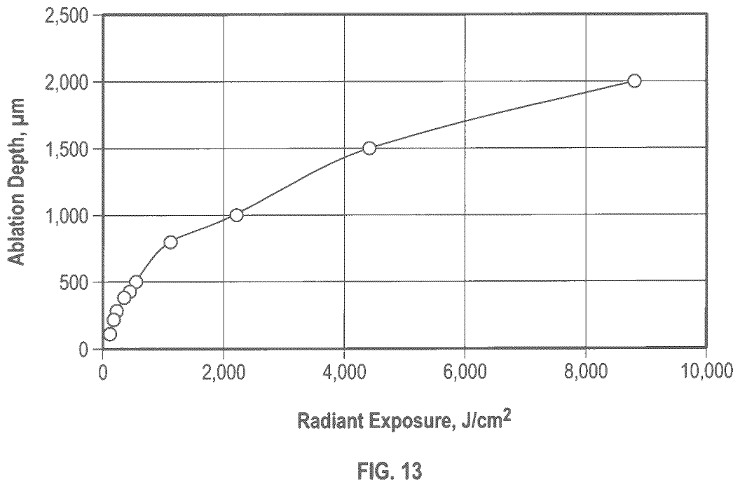

FIG. 13 is a graph showing ablation depth as a function of the radiant exposure for both superficial and deep ablation columns with 3.05 μm and 3.20 μm wavelengths and a 34 μm spot size. The results indicate that as radiant exposure increases, the ablation depth also increases. In accordance with common understanding, the spot size will not influence this relationship to any significant degree.

### Other Examples of Laser Parameters

According to some embodiments, the laser parameters (also referred to as laser operating parameters) include: a pulsed mode having a pulse duration within a range of 1 microsecond (ρs) to 250 milliseconds (ms) inclusive, and a duty cycle within a range of 0.1% to 50% inclusive. In some embodiments the duty cycle is within a range of 5% to 90% inclusive. This is also one example of where a laser parameter may be modulated by a controller.

### Added Coagulation to the Ablation Channel

In accordance with at least one embodiment, performing laser treatments using the wavelength range of 3.0 μm to 3.25 μm yields a coagulation zone within a range of 20 μm to 60 μm (inclusive). In accordance with at least one embodiment, a method is disclosed wherein an extended coagulation zone is achieved at the bottom of the ablated channel. This is accomplished in certain embodiments by adding multiple pulses over a longer period of time at a low RE. According to one example, a laser having a wavelength in the aforementioned range may have the following attributes: continuous-wave (CW) power of 0.5 to 2.0 W, with multiple pulses such as 10 pulses, each with pulse duration of 1-5 milliseconds (ms) and duration between pulses of 1-50 ms (in one example), and 1-5 ms (in another example). According to some embodiments, this extended coagulation is applicable for columns.

### Flat Top Super-Gaussian Beams

In accordance with one embodiment, the laser beam is incident on a surface of the biological tissue with a spot having an intensity profile that is a quasi-Gaussian profile or a flat-top profile. In Gaussian beams, the intensity smoothly decays from its maximum on the beam axis to zero. In certain embodiment, a quasi-Gaussian beam is used. In other embodiments, a flat-top beam is used, where the beam has an intensity profile that is flat (“rectangular”) over most of the covered area. The flat top beam still has smooth edges and it can be approximated with a supergaussian profile. In certain embodiments, such a supergaussian beam is used.

### Bessel Beams

In accordance with some embodiments, Gaussian beams (M² of 1-1.2) are used to perform laser treatment. Other types of beams may provide certain advantages, including use of a Bessel-Gauss beam which is used in accordance with one or more embodiments.

Ashforth, et al. (Ashforth, Oosterbeek, and Simpson, “Ultrafast pulsed Bessel beams for enhanced laser ablation of bone tissue for applications in LASSOS,” Proc. SPIE 10094, Frontiers in Ultrafast Optics; Biomedical, Scientific, and Industrial Applications XVII, 100941O (22 Feb. 2017); https://doi.org/10.1117/12.2250068) have discussed the significant decrease in the ablation thresholds as well as higher ablation efficiency with Bessel beams for bone ablation application. This has been explained by the ability of Bessel beams to remain focused for distances that are orders of magnitude greater than the Rayleigh range and their ability to self-reproduce despite obstructions along their propagation path.

In accordance with at least one embodiment, this concept is expanded for use of a Bessel beam in skin and cartilage laser treatment. In some embodiments, deep structures (e.g., >3 mm depth) such as cartilage and bone are treated. Fractional treatment is also disclosed. For non-invasive treatments, with focusing, superficial structures can be spared. Ablation damage will occur deeper where the focused radiant exposure exceeds the ablation threshold. Thus, non-invasive, fractional treatments of deeply situated structures such as cartilage and bne are disclosed.

According to various embodiments, approximations to Bessel beams are made in practice either by focusing a Gaussian beam with an axicon lens to generate a Bessel Gauss beam, by using axisymmetric diffraction gratings, or by placing a narrow annular aperture in the far field.

Bessel Beam with a Scanned Spot for a Line

In one embodiment for lines, the maximum dwell time is in the central axis along the line in the direction of scanning (call it along an x-axis).

Along the central axis (theta=0), RE=RE0=P/(d*v), x-direction, along motion.

For a flat-top bean, cosine distribution, RE=RE0*cos (theta), where RE0=P/(d*v). This effect is exaggerated with a Gaussian beam where the irradiance drops off to the edges of the circular spot.

According to some embodiments, a ring shaped beam profile is used. When scanned, this beam was found to yield a more uniform beam profile in the y-direction (perpendicular to motion-direction). Such beams can be called Bessel beams, obtained with axicons, with conical surfaces. These can also be obtained by a combination (e.g., two) of diffractive optical elements (DOE).

According to another embodiment, the beam profile is configured such that the ring is oval. The oval is configured such that the dip in the middle is in the y-direction only (when the direction of motion is in the x-direction). This has been found to give close to uniform RE with motion.

Coagulation with Exposure Using 1.56 μm Wavelength Irradiation of Skin

At 1.56 μm, the water absorption coefficient is 10 cm⁻¹. In accordance with certain aspects, the absorption coefficient of skin is estimated as 70% of this value: 7 cm⁻¹. In one embodiment, this wavelength yields a penetration depth of about 500 μm with pulse durations in the range of 0.1 to 10 ins, and results in a coagulation width in the range of 100-200 μm. This level of coagulation is desirable in certain applications, especially applications where skin contraction and tightening is a desired end-point.

According to one embodiment, an optical parametric oscillator (OPO) is pumped with a 1.03 μm laser (e.g., at a power of 50 W) and a signal at 1.56 μm (e.g., at a power of 1.5 W) laser. In some embodiments, at least a portion of the laser radiation emitted from the OPO is returned to the console and dissipated as heat. For instance, at least a portion of the pump beam exiting the OPO may be returned to the console and dissipated as heat. In some embodiments, at least a portion of the 1.56 μm signal beam is passed through to the target skin to cause coagulation. In some embodiments, this is implemented using appropriate choice of mirror coatings.

### Laser Channels Assisted Drug Delivery

Ablative fractional resurfacing has been used to increase the rate and amount of drug delivery into the dermis, in which the ablative holes bypass the stratum corneum barrier to the transport of the drug. This is especially true for large molecules, which have a very small diffusion coefficient through the stratum corneum. With disclosed embodiments of microfractional (small spot size and high number density) treatments, higher rates of drug transport are possible.

According to one embodiment, drug delivery through cylindrical surfaces with columns is implemented. In accordance with certain aspects, the mass transfer rate of drug into skin after microfractional treatment is proportional to the product of the surface area of the individually ablated cylinders and the number density of the cylinders on the surface. If one assumes that mass transfer through an ablated column of circular cross section happens principally through the cylindrical surface, the mass transfer area per skin surface area is given by 4*(coverage rate) ablation depth/hole diameter. For the same number density (or alternatively coverage rate) and the same ablation depth, the mass transfer area per skin surface is inversely proportional to the hole diameter, which is directly related to the spot size. Thus, the small spot size (≤45 μm) of the microfractional treatment coupled with the high number density allows for very high available area of mass transfer.

Tattoo Removal with Fractional Ablative Technology with Minimal Downtime

There is a need for more effective and more efficient (than existing) methods for removal of undesired tattoos. Laser pulses with pulse durations in the nanosecond and picosecond domain have conventionally been employed for tattoo removal treatments. The hypothesized mechanism is that the laser pulses break down the ink with the small pulse durations, which creates inertial and thermal confinement. The broken and smaller ink particles are removed from the skin by the lymphatic system. However, there is a need for quicker removal of such particles, and the aforementioned mechanism is inefficient and takes multiple (e.g., 10) treatments to obtain substantial clearance of the tattoos.

In accordance with certain embodiments, one or more fractional treatment methods are implemented after the “conventional” ns, ps, or even fs laser treatments. In one embodiment, a cup with suction is also used after the fractional treatment. This combination of treatments enhances tattoo clearance in at least the following two ways:
1, removal of parts of skin via ablation (the skin contains the ink, so some ink is removed).
2. removal of interstitial fluid that contains some of the broken ink particles.

In accordance with at least one embodiment, an intermediate step using urea is implemented between step 1 (fractional treatment) and step 2 (suction cup use) described above. The urea softens and dissolves part of the skin, thus making the transport of ink and fluid easier. According to a further embodiment, vibration is added before, during or both during application of suction. In another embodiment, fractional treatment is done prior to the nanosecond, picosecond, or femtosecond laser tattoo removal treatment. In accordance with at least one embodiment, it is believed that “microfractional” treatment (spot size ≤45 μm) would have better efficacy and quicker healing for tattoo ink removal compared to fractional treatment (spot size ≥120 μm).

### Minimally Invasive Fractional Laser Treatment of Deep Fascia and Periosteum

In accordance with one embodiment, the laser characteristics of the disclosed laser system allow for treatment of deep facial subcutaneous structures such as deep fascia and periosteum. Degradation of these structures with age is the major cause of facial skin sagging and sub-optimal aesthetic appearance. At present, there are no known minimally invasive interventions capable of ameliorating these conditions. Hence, such fractional treatments have a potential of radically improving outcomes of anti-aging facial procedures and providing unique benefits for patients suffering from facial skin sagging.

In accordance with certain embodiments, success of the deep fractional procedure depends on the following conditions, which include one or more embodiments:
1. Providing a laser system that is configured to produce ablative columns with aspect ratio >40 (preferably, >50) and surface diameter in a range of 70-90 μm inclusive. In one embodiment, this can be achieved by providing a laser system that is configured to generate pulse energies in a range of about 70-90 mJ and either regular (Gaussian) or Bessel optics (i.e., beam shape or profile).
2. Precisely controlled coagulative margin having a width in a range of 10 to 100 μm (provided by a laser system in accordance with the operating parameters described herein).
3. Adequate tissue cooling, preventing bulk heating and confluence of thermal damage from neighboring columns. Such cooling may be passive or active. In one embodiment where active cooling is desired, cooling is accomplished by blowing via cold air on the tissue surface.
   Maximizing Clinical Efficacy with Acceptable Discomfort and Acceptable Side Effects Profile
   Columns with Spots on Surface

As discussed previously, confluent or fully ablative treatments generally lead to high clinical efficacy but are beset with high pain and long healing times (and some probability of post-inflammatory hyperpigmentation (PIH) and scarring). With fractional ablative treatments as described herein, it is proposed that a maximum density of spots or lines should be used that have acceptable discomfort and acceptable healing time and other side effects such as PIH. The testing and results are described in further detail below.

According to one embodiment, ablative columns protruding into the skin are used (with spots on surface) as one example. The concept can also be extended to lines in accordance with other embodiments. Two kinds of treatments are considered, aesthetic and medical. The maximum density is typically lower for acceptable pain as compared to acceptable other side effects such as scarring, hematoma, and long term PIH. For aesthetic treatments such as improvement in wrinkles and fine lines, the maximum density of the dots or spots (number of dots per square centimeter) is limited by the discomfort felt by the patient during treatment. For medical treatments, for example, improvement in the appearance of burn scars, traumatic injury scars, or acne scars, a higher discomfort is acceptable to the patients. Then, the maximum density is limited by the acceptable side effects profile that can include scarring and/or the appearance of PIH.

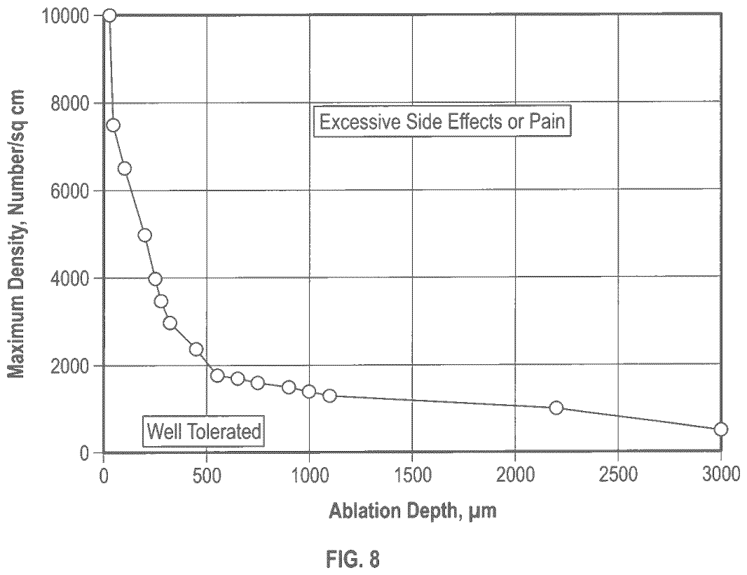

Such a determination of the maximum density of the dots (ablative columns in skin) was performed for various ablation depths in experiments conducted by the Applicant. Five subjects were treated on the thigh and forearm in a two-dimensional matrix format with a 34-um spot size device. The pulse energy was varied to obtain various ablation depths. For each ablation depth, a range of number densities were used. The subjects were asked for pain at each combination on a scale of 0-5 with 0-3 considered “tolerated” and 4, 5 as “not acceptable.” The follow-up point for side effects was 7 days post treatment. The side effects followed were: erythema, edema, hematoma, scarring, post inflammatory hyperpigmentation, or other unexpected side effects. For each ablation depth, the highest number density tolerated was identified for each of the above criteria. Then, the lowest number density (a conservative choice) was chosen as the maximum number density tolerated. These results are shown in FIG. 8. Thus, this data gives the acceptable, tolerated, and suggested upper limit for the number density for the microfractional treatment.

These number densities can be several thousands of spots per square cm at superficial ablation depths with a spot size less than 45 micrometers and are an important component of microfractional treatments. According to one embodiment, the injury pattern is an array of spots having a number density with a range of 100 spots/cm² to 10000 spots/cm² inclusive. In another embodiment, the number density is within a range of 150 spots/cm² to 1000 spots/cm² inclusive. According to one embodiment, the laser system (described in further detail below) is configured to generate pulsed radiation and the injury pattern includes ablation columns having a column density (defined as a number of columns per square centimeter of biological tissue) and the laser beam (discussed in further detail below) delivers pulsed laser radiation such that the column density has a maximum value within a range of 500 to 10000 inclusive for respective ablation depths within a range of 3000 μm to 25 μm inclusive. In another embodiment, the column density has a maximum value of 10000, 7500, 6500, 5000, 4000, 3500, 3000, 2500, 1800, 1700, 1600, 1500, 1400, 1300, 1000, and 500 for ablation depths of 25, 50, 100, 200, 250, 300, 350, 450, 550, 650, 750, 900, 1000, 1500, 2000, and 3000 μm respectively. With the density close to the maximum values, the efficacy will come close to efficacies obtained with fully ablative treatment but without the long healing times and side effects. Very high efficacy, i.e., improvement in skin texture, such as improvement of 2 or more in the Fitzpatrick wrinkle score (Fitzpatrick, et al., Pulsed carbon dioxide laser resurfacing of photo-aged facial skin, Arch Dermatol., pp 395-402, 1996) have been noted when number densities close to the maximum are used. Furthermore, in accordance with various embodiments, there is a minimum number density below which the efficacy is inadequate. In one embodiment, the column density has a minimum value with a range of 300 to 1300 inclusive for respective ablation depths within a range of 3000 μm to 25 μm inclusive. In another embodiment, the column density has a minimum value of 1300, 1200, 1100, 1000, 1000, 1000, 1000, 1000, 1000, 1000, 900, 800, 700, 600, 500, and 300 for ablation depths of 25, 50, 100, 200, 250, 300, 350, 450, 550, 650, 750, 900, 1000, 1500, 2000, and 3000 μm respectively. According to a further embodiment, intermediate values for the column density and ablation depth are obtained by interpolating between adjacent column density and ablation depth values.

Conventional ablative fractional treatments, as previously discussed, use larger spot sizes and have much lower number densities. One such system includes the Sciton ProFractional® XC, which uses an Er:YAG laser, a 430 μm spot, and a treatment density that can be set at 5.5%, 11% or 22%. The maximum suggested density of 22% and a spot size of 430 μm translates to a number density of 151 spots per square cm. Another example is the Lumenis UltraPulse®, CO₂ laser. Two spot sizes are available, 1.3 mm with ActiveFX™ and 120 μm with DeepFX™. With 1.3 mm, the theoretical maximum number density is if the pitch is sized to be the same as the spot diameter, and this density if about 60 spots per square cm. With 120 μm, Ramsdell (Ramsdell, 2012, Fractional Carbon Dioxide Laser Resurfacing, Semin Past Surg, vol 26, pp 125-130, https://www.ncbi.nlm.nih.gov/pnc/articles/PMC3580980/) suggests a maximum coverage rate of 15%. With a spot size of 120 μm, this translates to a number density of 2210 spots per square cm. A coverage rate of 25%, which is available on the device, translates to a number density of 4421 spots per square cm. The microfractional ablative laser systems and methods disclosed herein that implement a spot size smaller than 45 μm, use much higher number densities that have been shown to be tolerated and feasible.

### Linear Grooves on Surface

The injury pattern used to achieve the results described above is columns with spots on the surface of the biological tissue. For these, the pitch is the center-to-center distance between adjacent spots (assuming a square array). The coverage rate is (area of ablation zone/(pitch²)). The number density is (1/(pitch²)).

Similar analysis can be done for lines where linear grooves extending into skin are made. One non-limiting example of such a pattern is shown in FIG. 3A. According to at least one embodiment, the ability to determine the maximum coverage rate in this situation is provided. For a given velocity, power, and ablation diameter of the channel, the peak radiant exposure in accordance with one embodiment is calculated as power/(diameter_ablation*linear velocity). The pitch is defined as the center-to-center distance between adjacent lines. The coverage rate is defined as the ratio (ablation diameter divided by pitch). The density is defined as the number of lines per length-in-perpendicular direction or (1/pitch)).

As previously mentioned, the pitch may be defined as the center-to-center distance between two adjacent spots or two adjacent lines. In some embodiments, the pitch is sized to be in a range of 100 μm to 1 mm inclusive.

### Mid-IR Laser System Based on Difference Frequency Generation (DFG)

In accordance with at least one embodiment, a laser system is provided that is configured to provide difference-frequency generation outside the laser cavity, and in particular, provide a configuration of a mid-infrared laser light source using difference frequency generation of fiber laser radiation.

### Problem

High average power mid-IR radiation sources are suitable for a variety of applications, such as organic material processing, surgery, cosmetology, dentistry, etc. However, such conventional multi-watt level sources have significant drawbacks that limit their scope.

For instance, most of these devices are relatively large, which complicates the delivery of radiation to the processing area. The active element of gas in CO₂ lasers has a typical size about one meter in order to obtain sufficient gain and high enough power due to the low density of the gain medium. Active elements of solid-state (e.g., quantum and intraband cascade; bulk solid state such as Er:YAG, Cr:ZnSe, Ho:YAG, Ho:YLF and other; Thulium, Holmium and Erbium doped fiber) lasers require water cooling. As a result, it becomes impossible to place such radiation sources in a compact, ergonomic design, such as a handpiece.

For near-infrared lasers, this problem is resolved by using silica fibers to deliver radiation to a processing area. However, the delivery fibers for the mid-IR radiation are expensive ($100-$1000 per meter) and have poor characteristics (fragile, not suitable for cleaving and fusion splicing) due to the physical properties of transparent materials in this spectral range. This often necessitates the use of articulated arms to carry the mid-IR radiation from the laser to the skin target. The articulated arms are bulky, impractical, and prone to misalignment upon impact in common use. In accordance with one embodiment, radiation is directed with flexible silica fibers, which is advantageous over the expensive, fragile fibers as well as the use of an articulated arm.

It is also often impossible to obtain a sufficiently small size beam waist for such sources due to a large M² value or a long wavelength of radiation. This limiting factor can be critical for material processing because of the inability to obtain high power density and precise shape of the cut. Powerful solid-state based lasers are emitting multimode radiation due to the necessity of enlargement of an active area. Also, most of the fibers for mid-IR are configured for multimode radiation only. The wavelength of gas CO₂ lasers radiation also lies on the far edge of the mid-IR range.

The absorption coefficient of water is lower by one order of magnitude for the radiation of CO₂ lasers configured with a typical wavelength of 10.6 μm than it is for lasers configured to emit radiation near the 3 μm spectral range. This is important in applications targeting high water content materials, e.g., biological tissues.

### Laser System Example

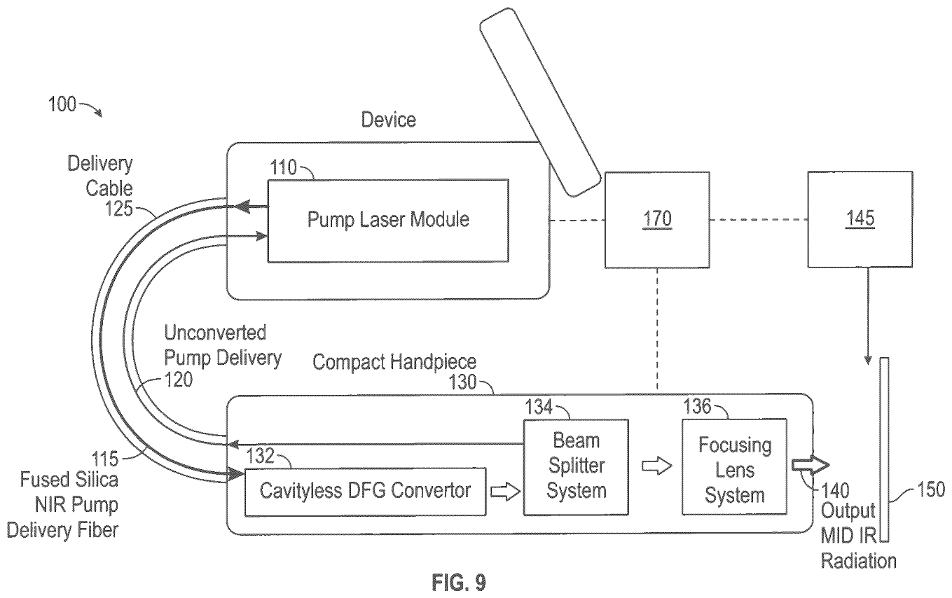

One non-limiting embodiment of a laser system suitable for the treatment methods disclosed herein includes a compact air-cooled mid-IR laser system that is based on nonlinear frequency conversion of near IR pump radiation from fiber laser in nonlinear optical crystals. A schematic representation of one example of a laser system 100 is shown in FIG. 9 in accordance with one embodiment. The general optical schematic shown in FIG. 9 reflects a hybrid laser configured with a compact wavelength converter.

Laser system 100 generally comprises a laser module 110 that comprise at least one laser source, a difference frequency generator 132 located with a handpiece 130, and an optical fiber 115 coupled to the laser module 110 and the difference frequency generator 132. In accordance with some embodiments, the difference frequency generator 132 is an optical parametric oscillator (OPO). In some embodiments, optical fiber 115 may be included in a fiber optical delivery cable 125. In some embodiments, the optical fiber 115 is silica fiber. According to at least one embodiment, radiation from the fiber laser(s) of the laser module 110 is transmitted to the handpiece 130 by a delivery fiber with a core diameter in a range of 10-90 μm inclusive.

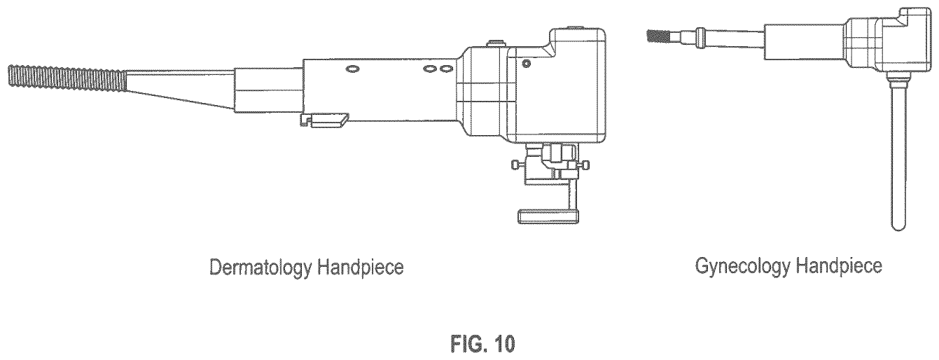

The handpiece 130 (also referred to herein as a compact handpiece) includes several components and is configured to output a laser beam 140 of laser radiation that may be used to perform laser treatment on the biological tissue 150. In some embodiments, the handpiece 130 has dimensions of about 20×200 mm, and a weight <0.2 kg. Perspective views of two non-limiting examples of a dermatology handpiece and a gynecology handpiece are shown in FIG. 10. In one embodiment, the handpiece 130 comprises a wavelength converter 132 (i.e., difference frequency generator) without the resonator inside. According to at least one embodiment, at least a portion of laser radiation emitted from the difference frequency generator 132 is directed back to the laser module 110. For example, in some embodiments, at least a portion of the radiation that is not converted to mid-IR is removed from the handpiece 130 by a dedicated silica fiber 120 (which may be included in delivery fiber 125) and terminated in the laser module 110. The laser module 110 is connected to the handpiece 130 by the delivery cable 125, which contains one or more silica fibers, electric cables and a protective hose. High conversion efficiency is achieved by using the near IR signal radiation for difference frequency generation. In some embodiments, a beam quality factor M² is close to 1 and determined, in general, by beam quality of the pump and the signal beams.

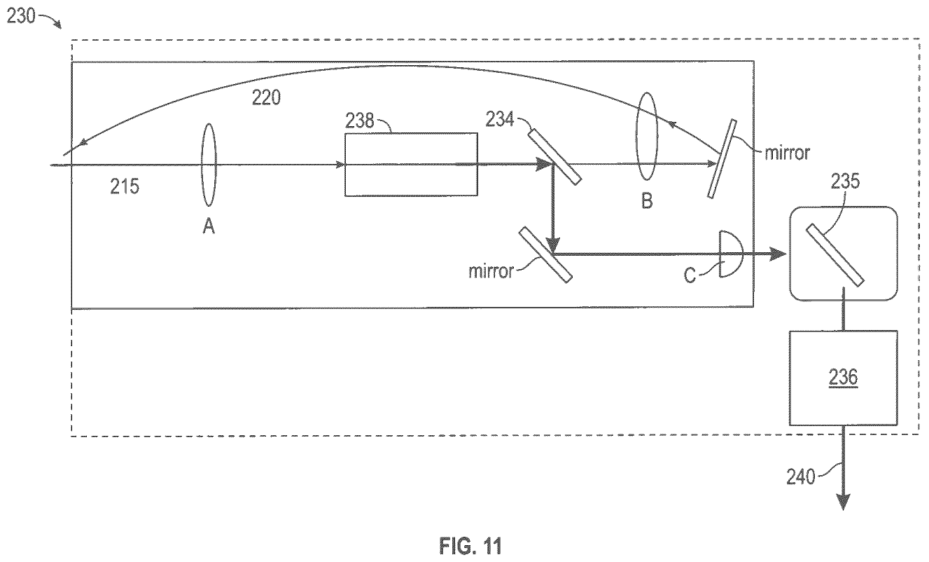

Laser system 100 also includes a controller 170. In some embodiments, the controller 170 is coupled to the laser module 110 and the handpiece 130 (and one or more components of the handpiece 130). In some instances a console may house one or more components of the laser system, such as the controller 170 and/or the laser module 110. The controller 170 is configured to scan the laser beam 140 over the biological tissue 150 in an injury pattern, where the injury pattern has a pitch. For example, controller 170 may be configured to control a scanner (e.g., scanner mirror 235 of FIG. 11 described below). As previously mentioned, the scanner is configured to create the injury pattern on the biological tissue.

The controller 170 includes circuitry that may be separate or integral components. It will be appreciated by those skilled in the art that the operations performed by the controller 170 may be performed by one or more controllers, processors, and/or other electronic components, including software and/or hardware components. For example, controller 170 includes a processor (which may include more than one processor) and a computer-Tradable-storage device, and a memory (also referred to as a storage device), as well as other hardware and software components as will be appreciated by those of skill in the art.

Generation schemes based on conversion in a nonlinear medium often use resonator cavities to achieve high-level conversion efficiency. However, this approach makes the implementation of such a converter into a compact robust design difficult and requires more sophisticated mechanical and optical construction elements.

In accordance with one embodiment, the laser module 110 comprises two fiber lasers arranged in a Master Oscillator Power Amplifier (MOPA) configuration. In some embodiments, the two fiber lasers are configured with 1.03 μm and 1.5-1.6 μm (e.g., 1.56 μm) wavelengths respectively, which are used as pump and signal, respectively, for difference frequency generation. According to at least one embodiment, the laser module 110 is configured to generate pulsed laser radiation. In one embodiment, the near-IR radiation pulses of the lasers in the laser module 110 have a duration of about 1-2 ns and are synchronized in time. In one embodiment, radiation from the diode pumped fiber lasers may be coupled into single mode (SM) silica fibers. As an example, radiation from each fiber laser source can be combined using a combiner (e.g., a wavelength division multiplexing (WDM) device in laser module 110) and output into a single fiber. In some embodiments, the pump and signal radiations are delivered to the handpiece 130 by a single fiber (e.g., optical fiber 115). In some embodiments, each of the fibers that carry light from the respective fiber laser source is made of silica, as well as the fiber (e.g., optical fiber 115) that carries the combined wavelengths. In some embodiments, SM fiber delivers laser radiation emitted from each of the two diode pumped fiber laser sources into a multiplexer where the laser radiation is combined and delivered to the difference frequency generator 132 by the optical fiber 115. In accordance with at least one embodiment, optical fiber 115 is SM fiber.

According to one embodiment, the handpiece 130 also comprises a focus system 136 (also referred to herein as an optical focusing system) that includes one or more lenses and a beam splitter system 134. One non-limiting example of a handpiece in accordance with one embodiment is shown in the schematic representation of FIG. 11. Handpiece 230 of FIG. 11 comprises a difference frequency generator with one or more nonlinear optical crystals and in this example is configured as OPO 238. Laser radiation (e.g., pump and signal wavelengths) included in optical fiber 215 is passed through the OPO 238. Handpiece 230 also comprises a focus system 236, which is configured to focus the laser beam to a spot size or beam spot. In one embodiment, the optical focusing system 236 is based on a microlens having a focal length in a range of 3-5 mm inclusive, which provides a minimal distance between pump and signal beam waists (waist diameter approximately 100 μm). According to one embodiment, one or more of the nonlinear optical crystal(s) comprising the OPO 238 is configured with periodic ferroelectric domain structure. In some embodiments, handpiece 230 also includes a thermostat for the nonlinear optical crystals (not explicitly shown in FIG. 11).

As mentioned above, at least a portion of laser radiation (e.g., unconverted laser energy) emitted from the OPO 238 is directed back to the laser module 110, which may be delivered using optical fiber 220 (which in some instances is a dedicated silica fiber). Handpiece 234 also comprises a beam splitter 234 that functions as a mid-IR radiation filtration system based on one or more dichroic mirrors. In accordance with one embodiment, the two mirrors (as shown in FIG. 11) have high reflectively in the 3.0-3.2 μm wavelength range and high transmission at the 1.03 μm and 1.56 μm wavelengths. The mirror after lens B is highly reflective at 1.03 μm and 1.56 μm wavelengths and these wavelengths are returned back via an optical fiber 120, 220 to the laser module 110. In such instances the laser module 110 comprises one or more components that utilizes this unconverted laser radiation. For example, according to one embodiment, the portion of laser radiation directed back to the laser module 110 via optical fiber 220 is unconverted radiation that is directed onto a beam dump that is actively cooled with air. In some instances, the beam dump is also configured with power measurement capabilities.

Handpiece 230 also comprises a scanner 235, which in some embodiments is a single mirror scanner (as shown in FIG. 11), but in other embodiments may be a two-galvo system with two mirrors with motion in perpendicular directions. Laser radiation generated by the OPO is directed to a biological tissue target via laser beam 240. Handpiece 230 also includes one or more optical devices, includes lenses A, B and C and other optical devices, such as a mirror (labeled) as shown in FIG. 11.

In accordance with certain embodiments, two different schemes of difference frequency generation may be used:
1. A single-stage scheme carried out in one nonlinear crystal (e.g. PPLN or PPLT):

$$1030 \to 1560 + 3050 nm ( PPLN 30.3 / PPLT \sim 30.5 µm ) ,$$

where 1.03 μm provides the pump power, 1.56 μm provides signal radiation, and 3.05 μm laser radiation is generated that is used to treat biological tissue.
2. A two-stage scheme can be implemented in two separate crystals or one crystal with two periodic ferroelectric domain structures. Each stage has different periods of ferroelectric domain structure designed to achieve quasi-phase matching for each process:

$$1030 \to 1560 + 3050 nm ( PPLN 30.3 / PPLT \sim 30.5 µm ) ,$$

$$1560 \to 3050 + 3200 nm ( PPLN 34.7 / PPLT \sim 33 µm )$$

At the difference frequency generation process in the second crystal, the radiation at 1.56 μm acts as a pump for the 3.05 μm wavelength and another idle component at 3.20 μm. According to one embodiment, the two-stage difference frequency generation has a 50% pump conversion efficiency at 3.05 μm and 3.20 μm wavelengths. It is to be appreciated that although FIG. 11 indicates a single OPO, according to other embodiments a second OPO may be included.

The good beam quality of the mid-IR radiation generated allows input into a waveguide, a fiber, or focusing (e.g., using optical focusing system 136) to a small diameter. In one embodiment, the laser beam has an M² value in a range of 1.0 to 1.5 inclusive. In another embodiment, the laser beam has an M² value in a range of 1.0 to 1.3 inclusive.

In accordance with certain embodiments, the laser module 110 comprises two fiber lasers. According to some embodiments, the laser module 110 comprises two diode laser pumped fiber lasers. In one embodiment, a first of the two fiber lasers is configured to generate laser radiation having a wavelength within a range of 1.00-1.05 μm and a second of the two fiber lasers is configured to generate laser radiation having a wavelength within a range of 1.5-1.6 μm. In one embodiment, a first of the two fiber lasers is configured to generate laser radiation having a wavelength of 1.03 μm and a second of the two fiber lasers is configured to generate laser radiation having a wavelength of 1.56 μm. In some embodiments, the generated laser energy is delivered via two flexible fibers into a single fiber (shown as 115 in FIG. 9) that is within a flexible umbilical cord (delivery cable 125) and delivered to handpiece 130. In one embodiment, a laser beam(s) of laser radiation generated by the two fibers lasers has an M² value in a range of 1.0 to 1.5 inclusive. In another embodiment, the laser beam has an M² value in a range of 1.0 to 1.3 inclusive.

In one embodiment, optics are used to mix the two wavelengths in a two-stage PPLN/PPLT crystal OPO convertor (e.g., OPO 238). The laser beam from OPO 238 is collimated on a scanner, which in this example is configured as a mirror 235, which is then focused (e.g., using optical focusing system 136, e.g., lens or lenses 236) to the desired, small spot size on the skin plane 150 (i.e., target biological tissue). The unconverted radiation is sent back to the console via a silica fiber 120 (220 in FIG. 11) where it is dissipated. The temperature of the OPO crystal 238 is controlled via a thermostat and heater (not shown in FIG. 9 or 11). In accordance with various embodiments, all optics are coated for the wavelengths of interest including any aiming beam (e.g., red). In some embodiments AR coatings for maximizing transmittance through lenses and reflective coating of mirrors to maximize reflectance are also implemented.

In some embodiments, the spot size on tissue can be in a range from 25 μm to 120 μm inclusive, in some embodiments is in a range of 30 μm to 80 μm inclusive, and in some embodiments is in a range of 10 μm to 45 μm inclusive. In one embodiment, the spot size is within a range of 30 μm to 45 μm inclusive.

According to one embodiment, the OPO crystal 238 and the scanner 235 reside in the handpiece 230. In some embodiments, the handpiece 130, 230 is configured to have the following characteristics:
Shape: a shape of two tubes, at right angles to each other, is disclosed. The first tube comprises of input fibers, collimating and focusing optics, OPO crystal, followed by more collimating optics. An electronically controlled scanner mirror (e.g., 235) changes the direction of the laser beam by 90 degrees and the beam traverses the second tube. The collimating optics achieve a small spot size on the skin surface for dermatological procedures. According to some embodiments:
   Dimensions of tube 1: range 190 mm-300 mm
   Dimensions of tube 2: range 30 mm-100 mm.
Weight of the handpiece; range 200 gm-1,000 gm.

The configuration of the laser systems disclosed herein provide several advantages. One is that the lasers of the laser module 110 generate laser beams having an excellent beam quality factor (M² value close to 1) which means the laser radiation can be focused into a smaller beam size, which is needed at least in part to create the “microfractional” treatment that heals rapidly and allows a high number density and leads to the high efficacy disclosed herein. Secondly, silica fiber (e.g., fiber 115) is used to deliver the pump and signal laser radiation to the handpiece. This is in contrast to CO₂ or Er:YAG laser sources, for which an articulated arm or specialized non-silica fibers are typically required to deliver the energy to the scanner in the handpiece. Furthermore, the handpiece 130, 230 does not need cooling, since the OPO configuration does not generate heat. This makes the handpiece less expensive and smaller dimensions and weight can be used. As an example, conventional systems that use flashlamps in the handpiece require water cooling (or other coolant) that has to be delivered to the handpiece.

In accordance with another embodiment, cooling of the skin is also provided using a cooling device, shown generally as cooling device 145 in FIG. 9. Cooling of the skin has two functions. The first is to reduce pain felt by the patient during treatment. The second is to avoid any bulk heating of the skin between adjacent spots to a temperature (or higher) that can cause pain and burning, which is approximately 44-48° C. and higher. These or higher temperatures can cause side effects and severe pain. Cooling will not allow the untreated skin between spots to reach such temperatures and will reduce or eliminate “bulk heating” related side effects. Cooling by cooling device 145 can be performed by blowing cold air, blowing liquid cryogen that evaporates upon touching the skin, or a fluid (transparent to 3-0-3.2 μm radiation) cooling of a sapphire plate that is in contact with tissue. In some embodiments, the controller 170 is coupled to the cooling device 145 and is configured to control the operating parameters of the cooling device 145. Cold air is the preferred modality for its simplicity. For example, cold air from a commercial Zimmer CRYO 6 apparatus from Zimmer Medizin Systems, Irvine, CA can be used to cool the skin before, during, and/or after the treatment in a scanned area. According to some embodiments, the cooling air temperature can be in the range of −30° C. to 10° C., and the flow rate can be as high as 1000 liter/minute.

The scope of this disclosure also extends to gynecological applications. In some embodiments, a gynecological attachment is disclosed which is attached to the above described dermatological handpiece 130. The attachment is a cylindrical tube for insertion into the vagina. The small spot sizes of the aforementioned dermatological handpiece 130 are reimaged onto the vaginal surface by turning the beam 90 degrees. In some embodiments, a sapphire plate may be used for contacting the vaginal surface to keep the ablation debris out of the attachment.

### Scanning Techniques

In accordance with certain aspects, a “stamping” mode is disclosed for purposes of scanning and delivering the laser radiation to the biological tissue. In some embodiments, stamping includes stamping in adjacent areas and treating all of the desired skin areas without overlap. A “stamp” includes placing the handpiece 130, 230 on the skin and then initiating the scan process, wherein the laser beam is focused onto a spot and turned on for a certain amount of time. When the laser is turned off, the beam is moved to the next spot to be treated within the same scan area, where it is turned on again for the desired duration of time. The power and the pulse duration at each location determine the energy delivered to the spot. The pitch determines the x- and y-coordinates of various spots within a scan area, which is programmed into the scanner. After all the desired locations of spots are treated within a scan area for one “stamp”, then the operator moves the “scan area” to an area that is separate from the previously treated area. In certain instances no overlap is desired between adjacent scan areas, as well as no untreated areas between the adjacent scan areas.

In accordance with at least one embodiment, a “feathering” technique is implemented that allows for a degree of overlap between adjacent scan areas by the user. For instance, it is possible that due to inaccuracy in placement of the two adjacent scan areas that a slight overlap will exist along the edges and/or the corners of the scan areas. This can lead to overtreatment and result in undesired effects. To reduce this probability, “feathering” is suggested along one or more edges of the scanned area. According to various embodiments, the number density (inversely proportional to the square of the pitch) or the pulse energy is reduced along one or more edges of the scanned area to achieve this effect.

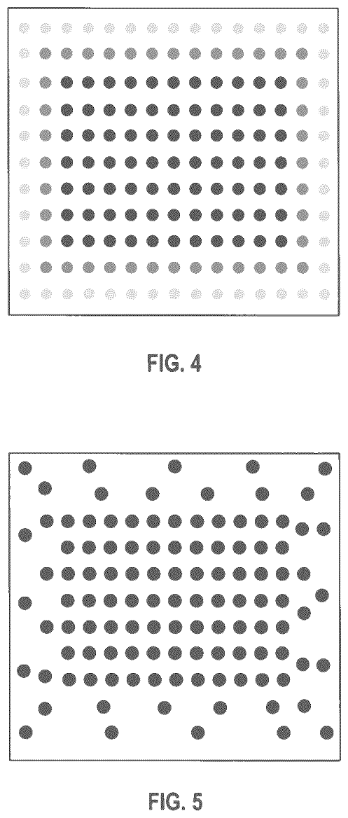

In accordance with at least one embodiment, the laser system 100 is configured to generate pulsed radiation, the injury pattern is an array of spots, and the controller 170 is configured to scan the laser beam 140 such that an RE per pulse is decreased on spots positioned near one or more edges of the array. A non-limiting example of such a technique is shown in FIG. 4, where an array of spots is shown, and the RE is decreased as indicated by gradual color lightening as one moves from the central portion of the array to the edges of the array (scanned area). In this instance, the pulse energy is decreased near the edges of the scan, as shown in the graph of FIG. 7.

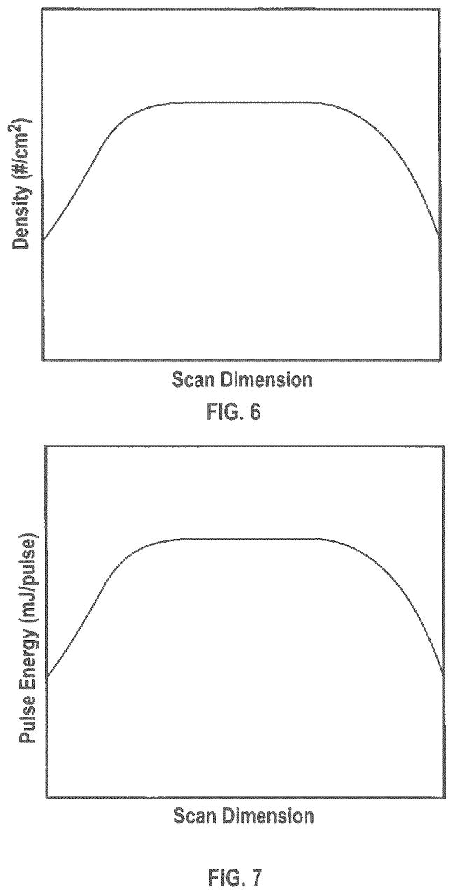

In some embodiments, the injury pattern is an array of spots and the controller 170 is further configured to scan the laser beam 140 such that a number density of spots is lower near one or more edges of the array. A non-limiting example of such a technique is shown in FIG. 5, where the number density of spots in the center is higher (i.e., the pitch is lower) than at the edges of the array (scanned area). FIG. 6 is a graphical representation of how the density decreases at the edges of the scan dimension.

According to some embodiments, a combination of the two “feathering” techniques described above may also be used.

### Clinical Testing Example

A subject was identified with significant per-auricular wrinkles. Four areas with wrinkles were identified in the peri-auricular skin area in front of the left ear. Based on the ablation depth—radiant exposure curve on ex vivo tissue, the following laser parameters were used with the microfractional treatment with the 3.05/3.20 μm wavelength ablative microfractional device with a 42 μm spot size. For each location, an area of 10 mm×10 mm dimensions was treated. Pain was deemed very tolerable.

| | Ablation | Energy, | Pitch, | number of |
|---|---|---|---|---|
| Area | Depth, μm | mJ | μm | spots/sq cm |
| 1 | 200 | 3 | 200 | 2601 |
| 2 | 300 | 5 | 200 | 2601 |
| 3 | 400 | 6 | 300 | 1179 |
| 4 | 500 | 7 | 400 | 676 |

Photographs of the treated areas were taken at baseline (before treatment), 2 weeks, and 5 weeks after treatment. The photographs were graded by an observer who was not aware of which areas got which treatment. The observer was asked to rate erythema, edema, and PIH at the follow-up on a scale of 0-3 (0: none, 1: mild, 2: moderate, 3: severe). The observer was also asked to rate the improvement in wrinkles on a scale of 0-3 (0: none, 1: mild, 2: moderate, 3: excellent improvement). The results at 5 weeks post treatment are as follows.

| Area | Erythema | Edema | PIH | Wrinkle Improvement |
|---|---|---|---|---|
| 1 | 0 | 0 | 0 | 3 (excellent) |
| 2 | 1 | 0 | 0 | 3 (excellent) |
| 3 | 0 | 0 | 0 | 2 (moderate) |
| 4 | 0 | 0 | 0 | 3 (excellent). |

This example demonstrates preliminary indication of excellent efficacy with minimal side effects.

The aspects disclosed herein in accordance with the present invention, are not limited in their application to the details of construction and the arrangement of components set forth in the following description or illustrated in the accompanying drawings. These aspects are capable of assuming other embodiments and of being practiced or of being carried out in various ways. Examples of specific implementations are provided herein for illustrative purposes only and are not intended to be limiting. In particular, acts, components, elements, and features discussed in connection with any one or more embodiments are not intended to be excluded from a similar role in any other embodiments.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated reference is supplementary to that of this document; for irreconcilable inconsistencies, the term usage in this document controls. Moreover, titles or subtitles may be used in the specification for the convenience of a reader, which shall have no influence on the scope of the present invention.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

What is claimed is:

1. A device for performing treatment of biological tissue, comprising:
a laser system configured to provide a laser beam having a wavelength within a range of 3.0 microns (μm) to 3.25 μm inclusive and a spot size within a range of 10 μm to 45 μm inclusive; and
a controller coupled to the laser system and configured to scan the laser beam over the biological tissue in an injury pattern, the injury pattern having a pitch that is sized to be in a range of 0.1 mm to 1 mm inclusive.

2. The device of claim 1, wherein the spot size is within a range of 30 μm to 45 μm inclusive.

3. The device of claim 1, wherein the laser system is configured to generate pulsed radiation such that a radiant exposure (RE) per pulse is within a range of 30 J/cm² to 6000 J/cm² inclusive.

4. The device of claim 3, wherein the RE per pulse is within a range of 100 J/cm² to 4000 J/cm² inclusive.

5. The device of claim 1, wherein the injury pattern is an array of spots or lines.

6. The device of claim 5, wherein the injury pattern is an array of spots on a surface of the biological tissue having a number density within a range of 100 spots/cm² to 10000 spots/cm² inclusive.

7. The device of claim 6, wherein the laser system is configured to generate pulsed radiation and the injury pattern includes ablation columns, the ablation columns having a column density defined as a number of columns per square centimeter of biological tissue, and
the column density having a maximum value of 10000, 7500, 6500, 5000, 4000, 3500, 3000, 2500, 1800, 1700, 1600, 1500, 1400, 1300, 1000, and 500 for ablation depths of 25, 50, 100, 200, 250, 300, 350, 450, 550, 650, 750, 900, 1000, 1500, 2000, and 3000 μm respectively.

8. The device of claim 6, wherein the laser system is configured to generate pulsed radiation and the injury pattern includes ablation columns, the ablation columns having a column density defined as a number of columns per square centimeter of biological tissue, and
the column density having a minimum value of 1300, 1200, 1100, 1000, 1000, 1000, 1000, 1000, 1000, 1000, 900, 800, 700, 600, 500, and 300 for ablation depths of 25, 50, 100, 200, 250, 300, 350, 450, 550, 650, 750, 900, 1000, 1500, 2000, and 3000 μm respectively.

9. (canceled)

10. The device of claim 5, wherein the laser system is configured to generate pulsed radiation, the injury pattern is an array of spots, and the controller is further configured to scan the laser beam such that a radiant exposure (RE) per pulse is decreased on spots positioned near one or more edges of the array.

11. The device of claim 5, wherein the injury pattern is an array of spots and the controller is further configured to scan the laser beam such that a number density of spots is lower near one or more edges of the array.

12. The device of claim 5, wherein each spot in the array of spots and each line in the array of lines has an ablation depth within a range of 25 μm to 3000 μm inclusive.

13. The device of claim 1, wherein
the laser system is configured to generate pulsed radiation such that each pulse has a peak power within a range of 0.1 W to 50 W inclusive, and
the laser beam is incident on a surface of the biological tissue with a spot having an intensity profile that is a quasi-Gaussian profile a flat-top profile, or a Bessel-Gauss profile.

14. (canceled)

15. The device of claim 1, wherein
the controller is further configured to control or modulate at least one laser parameter of the laser system, and
a laser source of the laser system is configured to operate in a pulsed mode and the at least one laser parameter includes
a pulse duration within a range of 1 microsecond (ρs) to 250 milliseconds (Ms) inclusive, and
a duty cycle within a range of 5% to 90% inclusive.

16-18. (canceled)

19. The device of claim 1, wherein the laser system comprises
a laser module comprising at least one laser source;
a difference frequency generator located within a handpiece;
an optical focusing system located within the handpiece and configured to focus the laser beam to the spot size; and
an optical fiber coupled to the laser module and the difference frequency generator.

20. The device of claim 19, wherein
the difference frequency generator is an optical parametric oscillator (OPO),
a laser beam of laser radiation generated from the OPO is directed onto a treatment area of the biological tissue, the laser beam configured to perform tissue ablation and coagulation, and
at least a portion of laser radiation emitted from the OPO is directed back to the laser module.

21-22. (canceled)

23. The device of claim 19, further comprising a scanner located within the handpiece.

24. The device of claim 19, wherein the laser module comprises two diode pumped fiber laser sources.

25. The device of claim 24, wherein single mode (SM) fiber delivers laser radiation emitted from each of the two diode pumped fiber laser sources into a multiplexer where the laser radiation is combined and delivered to the difference frequency generator by the optical fiber.

26. The device of claim 24, wherein laser radiation emitted from each of the two diode pumped fiber laser sources is mixed and delivered to the difference frequency generator by the optical fiber.

27. A method of conducting an ablative laser treatment on biological tissue, comprising:
generating a laser beam having a wavelength within a range of 3.0 microns (μm) to 3.25 μm inclusive and a spot size in a range of 10 μm to 45 μm inclusive; and
creating an injury pattern on the biological tissue with the laser beam.

28. The method of claim 27, wherein the spot size is within a range of 30 to 45 microns inclusive.

29. The method of claim 27, wherein the injury pattern includes ablation columns and the laser beam delivers pulsed laser radiation, the ablation columns having a column density defined as a number of columns per square centimeter of biological tissue, and
the column density having a maximum value of 10000, 7500, 6500, 5000, 4000, 3500, 3000, 2500, 1800, 1700, 1600, 1500, 1400, 1300, 1000, and 500 for ablation depths of 25, 50, 100, 200, 250, 300, 350, 450, 550, 650, 750, 900, 1000, 1500, 2000, and 3000 μm respectively.

30. The method of claim 27, wherein the injury pattern includes ablation columns and the laser beam delivers pulsed laser radiation, the ablation columns have a column density defined as a number of columns per square centimeter of biological tissue, and
the column density having a minimum value of 1300, 1200, 1100, 1000, 1000, 1000, 1000, 1000, 1000, 1000, 900, 800, 700, 600, 500, and 300 for ablation depths of 25, 50, 100, 200, 250, 300, 350, 450, 550, 650, 750, 900, 1000, 1500, 2000, and 3000 μm respectively.

31. The method of claim 29 or 30, further comprising intermediate values for the column density and ablation depth by interpolating between adjacent column density and ablation depth values.

32-43. (canceled)
